(19) United States
(12) Patent Application Publication
(10) Pub. No.: US 2005/0020934 A1
(43) Pub. Date: Jan. 27, 2005

(54) Physiological monitoring

(76) Inventors: Charles Potter, Standlake (GB); Nicholas McGrogan, Wallingford Oxon (GB)

Correspondence Address: NIXON & VANDERHYE, PC, 1100 N GLEBE ROAD, 8TH FLOOR, ARLINGTON, VA, 22201-4714, US

(21) Appl. No.: 10/494,311

(22) PCT Filed: Oct. 30, 2002
(86) PCT No.: PCT/GB02/04874

(30) Foreign Application Priority Data
Nov. 1, 2001 (GB) 01262864

(51) Int. Cl. A61B 5/0476; A61B 5/0488; G06F 17/00

(52) U.S. Cl. 600/546; 600/544; 128/920

(57) ABSTRACT

Sleep or vigilance analysis is performed by use of a trained artificial neural network on electro-oculogram signals. Either a left side or right side EOG signal may be used, referenced to the nearby mastoid site, the nape of the neck or another quiet site. The use of the EOG signal allows REM sleep to be distinguished from light sleep. The use of only one channel of data means that only a small number of electrodes need to be used and thus the device can be provided as a self-contained compact portable unit. The device may also provide a sleep stage type sensitive alarm clock.

The present invention relates to improvement in physiological monitoring, in particular sleep or vigilance monitoring.

Sleep studies currently involve the use of multiple electrodes connected to the subject to record the data necessary to make a diagnosis of a sleep disorder. A polysomnography study will typically require the following electrodes to be attached to the head, but may include many more:
Central EEG
Frontal EEG
Right EOG (Electro-Oculogram)
Left EOG
Right EMG (Electro-Myogram)
Left EMG
Right and/or Left Mastoid

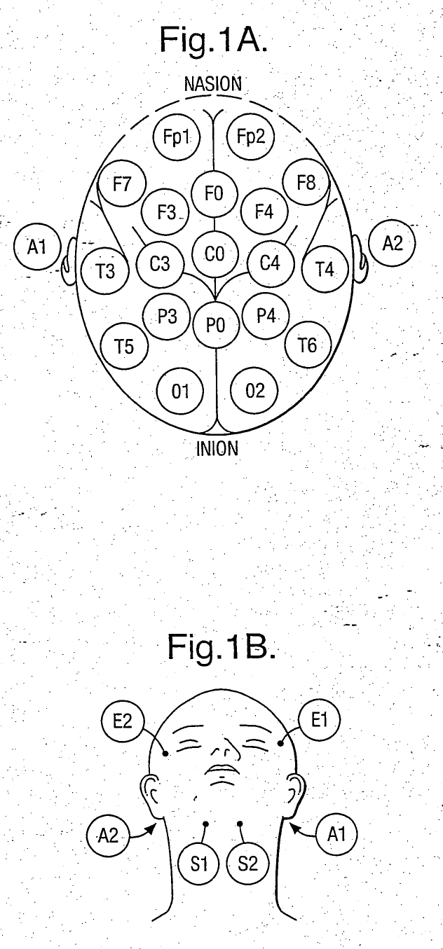

FIGS. 1a and 1b of the accompanying drawings show respectively the top and view from underside of a subject's head indicating the standard positions for locating electrodes for EEG monitoring. These locations are identified in FIGS. 1a and 1b by their commonly-used designations. Electrodes for electro-oculographic signals (EOG) are shown at position E1 located approximately 1 cm above and slightly lateral to the outer canthus of the left eye. Another EOG electrode is located approximately 1 cm below and slightly lateral to the outer can thus of the right eye at position E2. Positions A1 and A2 are the mastoid sites, located on the mastoid bone just behind the ear. Sites S1 and S2 are for electro-myographic signals.

Traditionally the right or left mastoid electrode has been used as a reference electrode for the others, though other “quiet” sites are possible such as the nape, of the neck.

To obtain fill information from the study the subject may also be required to have the following sensors attached:
ECG electrodes (typically three)
Thoracic respiration band
Abdominal respiration band
Throat microphone
Nasal/throat airflow
Leg sensors
Position sensor
Pulse oximeter

The data from the electrodes on the head is used to “score” the sleep stages of the patient throughout the recording. The method currently employed world-wide for scoring sleep recording is described in Rechtschaffen and Kales (1968), “A manual of standardized technology, techniques; and scoring systems for sleep stages of human subjects”. These are known as the R & K rules. Sleep scoring breaks the recording into epochs of typically 30 seconds duration and each epoch has a sleep stage classification applied to it. The six recognized classifications are: Stage wake; stage REM (rapid eye movement); stages 1, 2, 3 and 4. The classification of each epoch requires the identification of particular features in the EEG and EOG signals, and measurement of the amplitude of the EMG relative to the background EMG level. The features are identified using frequency and amplitude criteria. A set of rules is then applied to the features to obtain the classification for each epoch. Traditionally such a polysomnographic analysis is performed by a human expert visually inspecting the signal traces.

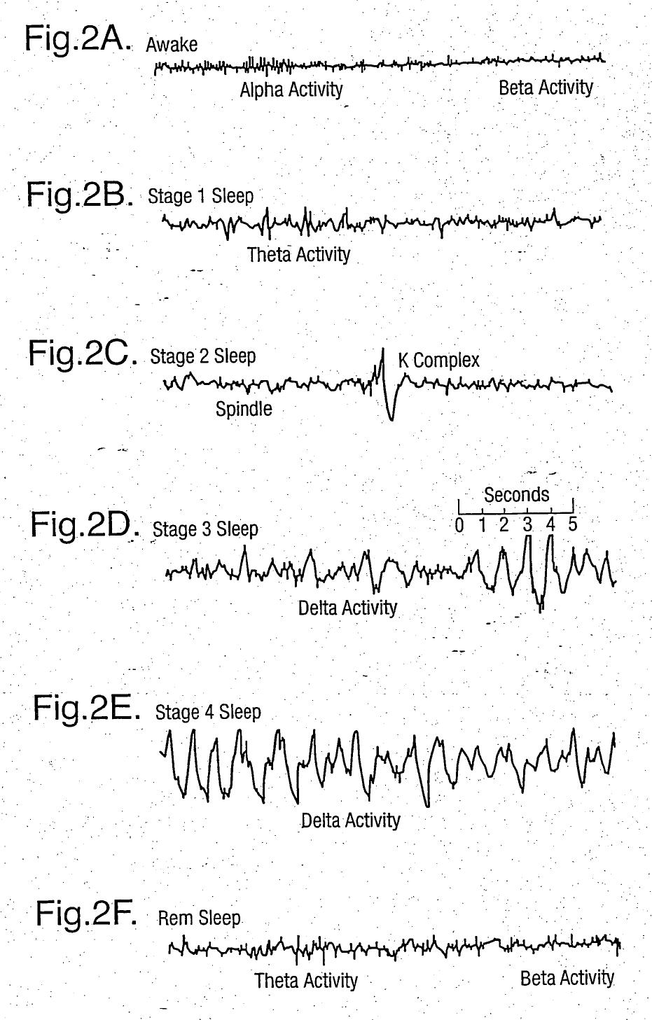

Examples of conventional EEG traces which have been assigned to: the sleep stages mentioned above are shown in FIG. 2a to 2f. FIG. 2a to 2f show the following stages:

FIG. 2a—awake;

FIG. 2b—stage 1;

FIG. 2c—stage 2;

FIG. 2d—stage 3;

FIG. 2e—stage 4;

FIG. 2f—REM.

Once each epoch has been assigned a classification, cleanup rules are applied that can reclassify certain epochs according to their context. The classifications of each epoch for the entire night's recording can be plotted against time. This is known as a “hypnogram”. Summary statistics can be derived from the hypnogram that allow objective measures of the quality of sleep to be made.

Thus the traditional methods of polysomnographic, analysis rely on human experts, however there have been proposals for automated analysis of sleep signals.

The article “A likelihood based computer approach to conventional scoring of sleep”, Procs. An. Int. Conf. of the Engineering in Medicine and Biology Society, 1992 Oct 29 to November 1, Vol. 14, pp 2645 to 2646 discloses a method of scoring sleep, using a computer program which divides each 30 second epoch into one second intervals and calculates the likelihood of the signal in each interval matching one of eleven pre-defined features. These likelihoods are then combined into the likelihood of each of eighteen events for the epoch. The signals are the conventional five channel signals: EEG, 2EOG, 2EMG. The R& K rules can then be applied to the events by combining their probabilities using a weighting matrix to assign a sleep stage type to the epoch.

A problem with the techniques mentioned above is that the patient needs many electrodes to be attached to the head. This is inconvenient, particularly where the analysis is being performed on a patient whose sleep is probably poor anyway. Having five or more electrodes attached to their head during sleep is likely to reduce the quality of their sleep even more. Further, similar techniques are used also in vigilance monitoring, which is analogous to sleep monitoring. In vigilance monitoring the same signals are monitored, but rather than sleep stage type, wakefulness types are assigned to each epoch. Thus, for instance, the vigilance of a, person operating machinery, driving or performing some safety-critical task, can be monitored. If the wakefulness stage types indicate a level of vigilance lower than required, then an alarm may be triggered. Again, the need to use five or more electrodes attached to the subject may hinder the activity of the subject. Further, movement of the subject in performing their normal activities can disturb the electrodes and cause noise in the signals which confuses the analysis.

EP-A-0773504 discloses a method and apparatus for sleep and vigilance monitoring in which a trained neural network is used to analyse the signal from the mastoid, sites of a subject's head. It can generate from this signal a hypnogram or series of wakefulness stage types, and can generate from these a summary of the quality of sleep, or trigger a vigilance alarm depending on the application. The use of only the mastoid sites is advantageous in reducing the number of electrodes which have to be attached to the subject. Analysis of the signals from the mastoid sites allow, in the case of sleep, the characterisation of each epoch into one of the following three sleep stage types: Wake, REM/light or deep. Thus it does not provide a characterization of all six sleep stage types, but nevertheless requires the use of fewer electrodes, and provides for automated real-time analysis.

According to the present invention there is provided sleep monitoring apparatus comprising one or more electrodes to obtain an electro-oculogram (EOG) signal from a subject over a period of epochs, the signal being related to the sleep stage type being experienced by the subject; and a processor adapted to analyse the electro-oculogram signal and assign a sleep stage type to each epoch.

The invention also provides in a corresponding way, vigilance monitoring apparatus comprising one or more electrodes to obtain an electro-oculogram (EOG) signal from a subject over a period of epochs, the signal being related to the wakefulness stage type being experienced by the subject; and a processor adapted to analyse the electro-oculogram signal and assign a wakefulness stage type to each epoch.

In particular the present invention provides for distinguishing between the different stages including between REM and light sleep by use of the EOG signal. Preferably the EOG signal alone is analysed, this involving use of one EOG electrode (attached to the left or right EOG site) and a reference electrode. The reference electrode may be connected to the nape of the neck, or one of the mastoid sites. It is particularly advantageous for the reference site to be the mastoid site neighbouring (ie on the same side of the head) as the EQG site. This allows a very convenient siting of electrodes.

Preferably the invention provides for the characterisation of each epoch as one of at least four types, namely wake, REM sleep, light sleep, or deep sleep, on the basis of the EOG signal alone.

In the case of sleep monitoring the process may be adapted to generate a hypnogram from the assigned sleep stage types, and to analyse the hypnogram to generate a summary index of sleep quality over the period of epochs. This summary index may be displayed on a display.

In the case of vigilance monitoring the apparatus may include a wakefulness stage type monitor for monitoring the assigned wakefulness stage types to determine whether they meet predetermined criteria which represent a lowered level of vigilance. The apparatus may also include a message generator responsive to the wakefulness stage type monitor to generate a message, such as an alarm, when the predetermined criteria are met.

Preferably the process comprises a trained artificial neural network. Preferably the apparatus is a single portable unit.

The process may be further adapted to analyse the EOG signal to derive a heart rate measurement therefrom. This heart rate measurement may be recorded and optionally displayed.

The invention will be further described by way of example with reference to the accompanying drawings in which:—

FIGS. 1a and 1b illustrate the standard sites for positioning electrodes on the head of a patient or subject;

FIG. 2a to 2f illustrate EEG signals and assigned sleep stage types;

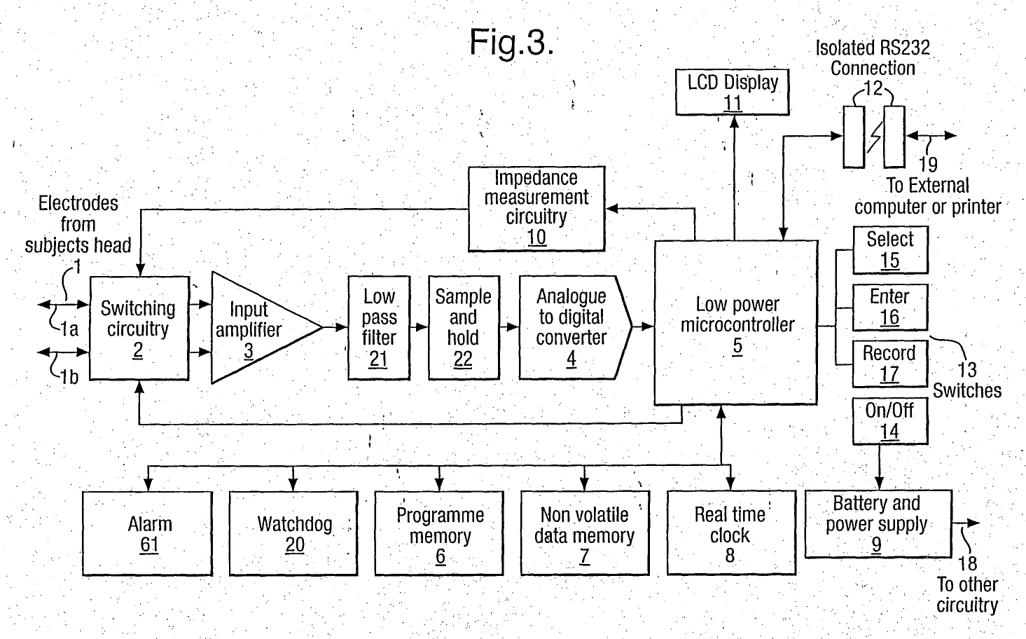

FIG. 3 illustrates a sleep and/or vigilance monitor apparatus in accordance with one embodiment of the present invention;

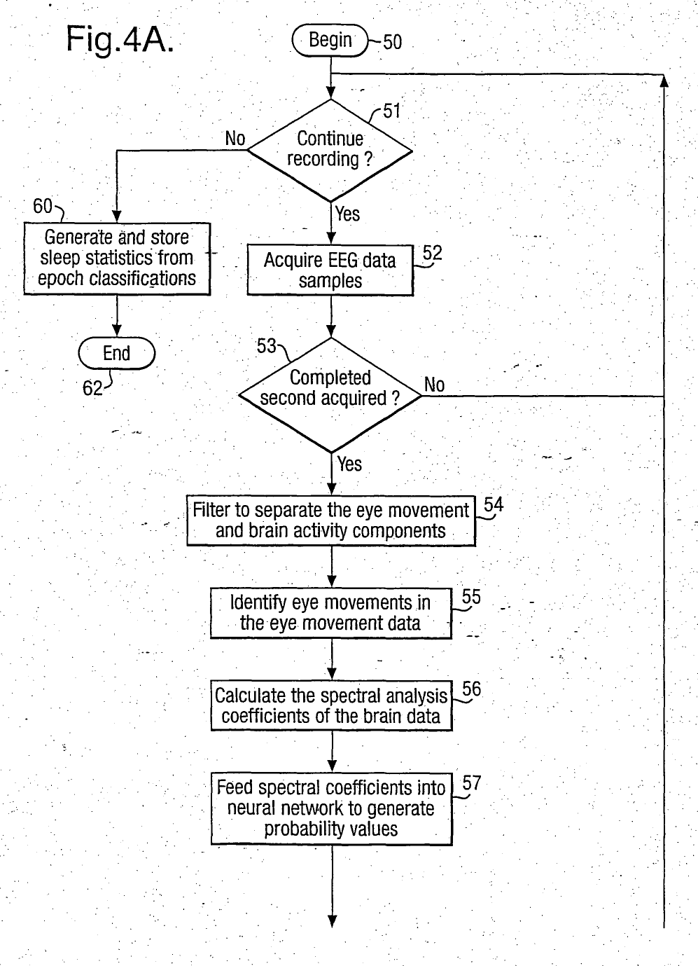

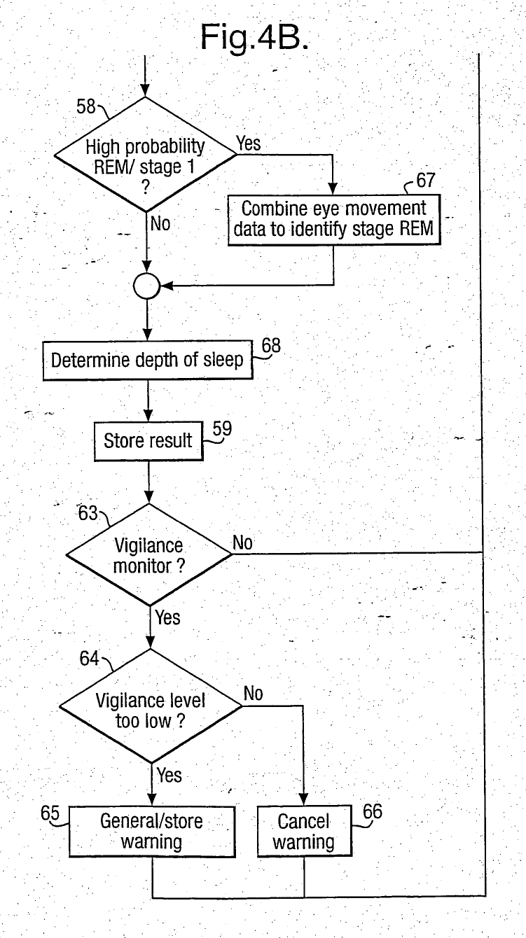

FIGS. 4A and B illustrates the process of analysis in one embodiment of the present invention;

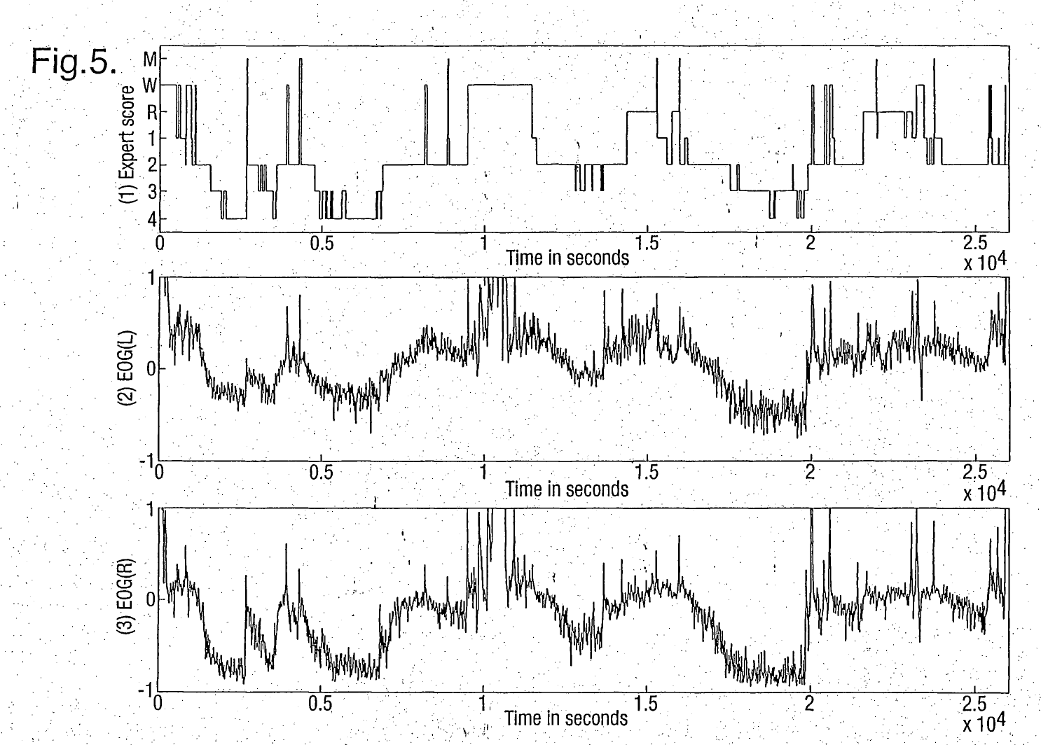

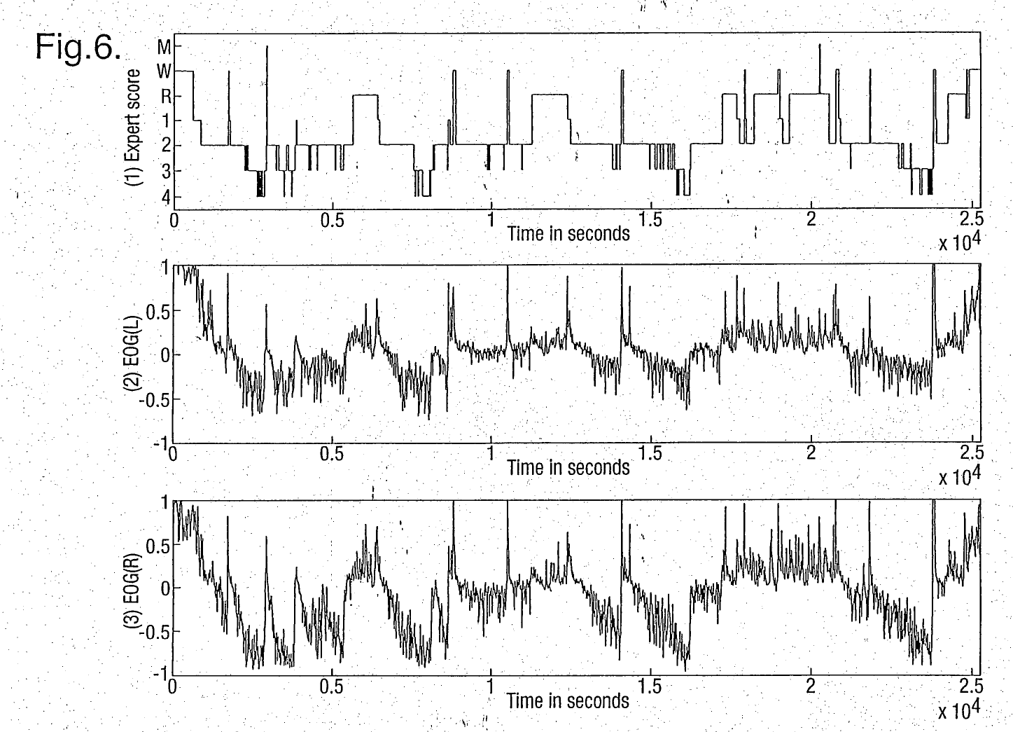

FIGS. 5 and 6 show hypnograms for a full night's sleep for two different subjects, trace (1) on FIGS. 5 and 6 show the expertly scored data for the fall nights sleep and traces (2) and (3) on FIGS. 5 and 6 show the data generated by an embodiment of the invention from the right and left EOG signals.

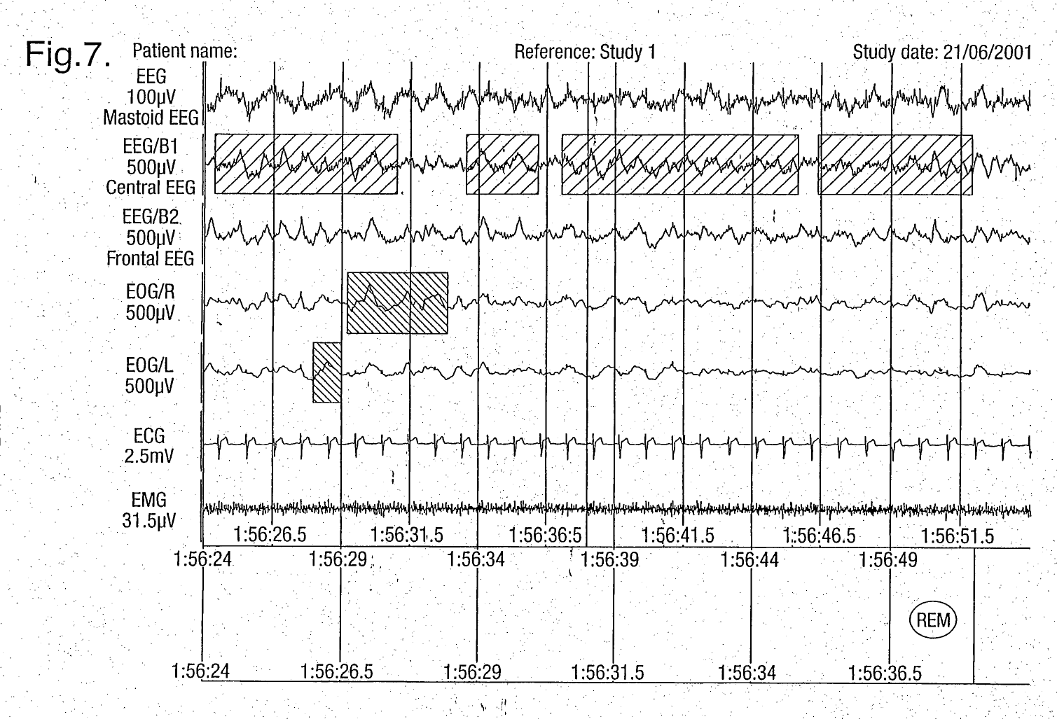

FIG. 7 to 10 show 30 seconds of raw data for several EEG channels for two different subjects in various sleep stage types.

A block diagram of a typical implementation of the device according to an embodiment of the invention is described below, which refers to FIG. 3. The device is a small, self-contained portable unit which can continually acquire and analyse signals from a patient for at least 12 hours. Results are held in nonvolatile data memory 7 for later display on LCD display 11 for downloading via an isolated RS232 or other link 12. The power is provided by internal primary or rechargeable batteries and a regulated power supply 9.

Signal are acquired from electrodes 1 mounted on the subject's head via switching circuitry 2 and input amplifier 3. The electrodes 1a, 1b and, where used, the indifferent electrode may be wireless electrodes thus making use of the device less obtrusive to the subject. The input amplifier has an analog bandwidth of at least 0.5 Hertz to 30 Hertz and is of a high gain, low noise instrumentation design. The signal is input to low-pass filter 21 to reduce unwanted aliasing components before analog to digital conversion using sample-and-hold circuit 22 and A/D converter 4. The resultant quantised data samples are transferred to low power microcontroller 5 for processing. In one example the sampling rate may be 128 Hertz and the quantisation of the analog to digital converter 4 be 12 bits, which provides sufficient dynamic range not to require a gain control on the input amplifier 3.

When recording one channel of EOG from the EOG site, three electrodes are typically necessary; two of them (1a, 1b) comprise the differential inputs to input amplifier 3 (one electrode being connected to an EOG site and one to a reference site such as the nearby mastoid site). The third is an “indifferent” lead (not shown) whose sole function is to allow input amplifier return currents to flow. The indifferent lead can be attached to any part of the subject's body. The indifferent lead is optional, it is possible to omit it.

Before signal acquisition begins, the impedances of the electrodes on the subject's head may be measured by causing impedance measurement circuitry 10 to drive a signal of known amplitude and source impedance via the switching circuitry 2 through the electrodes 1a, 1b in turn onto the subject's scalp. The resultant signal is measured by the microcontroller by the process described above and from it the impedances of the electrodes fitted to the subject's scalp are calculated in turn. A warning message is displayed on the LCD display 11 if the impedance of the connections to the subject's head is unacceptably high.

During data acquisition, the device continually acquires signals from the subject's head for analysis. The microcontroller 5 analyses the quantised values and from them generates results that are stored in non-volatile data memory 7. The program for the microcontroller is held in program memory 6. Real time clock 8 allows the results to be stored with a record of the time of day of acquisition.

Watchdog 20 resets the microcontroller 5 if the microcontroller fails to write to it periodically. If the microcontroller 5 is reset, it will identify whether it was in record before the reset was received and if so, go back into record so that a minimal amount of data is lost.

Alarm 61 may be activated if the device is a vigilance monitor and the processor 5 has determined that the level of vigilance of the subject is unacceptably low.

Control of the device is via switches 13, some of which can be read by the microcontroller 5. On/Off switch 14 turns the device on and off; select switch 15 displays successive prompts and results of the LCD screen 11; enter switch 16 accepts the command-currently displayed on the LCD screen 11; and record switch 17 puts the device into record which starts signal acquisition, processing and storage.

When the device is switched on the user can choose whether to view the results from a previous recording; down-load results from the previous recording into a computer or to a printer; delete the results from a previous recording; or record new data.

The low-pass filter 21 can be either part of the input amplifier 3 or can be achieved in the sampling process itself within the A/D converter 4. The sleep summary index, or other results of the lysis; can be displayed on the LCD display 11, though other types of display are possible. Further, data may be downloaded via the RS232 connection.

It will be appreciated that the above describes a specific embodiment and that variations are possible. For instance, the device may have only the means necessary to record results, with all analysis and display carried out externally.

The signals from the electrodes are processed by microprocessor 5 using a, neural network such as a multi-level perceptron (MLP). The neural network mar be trained in the same manner as described in EP-A-0773504 (which is hereby incorporated by reference) and the assignment to each epoch of a sleep or wake stage type is also carried but in the same way as described in BP-A-0773504. In EP-A-0773504 the neural network is trained to assign sleep stage and wake stage types on the basis of signal from the mastoid site. It is found that a neural network, trained on a central channel signal can be signal, can be used to assign sleep stage and wakefulness stage types on the EOG signal in accordance with the present invention without retraining However, it is also possible to train the neural network on the basis of the EOG signal directly in an analogous way to the training on the mastoid site in EP-A-0773504.

FIGS. 4A and B of the accompanying drawings illustrates a flow chart for the acquisition and processing of data.

Once put into record at step 50, after checking whether the recording must continue at step 51 the microcontroller acquires data from the EOG site at step 52 until it has one second's worth of data at 53. The data is filtered at step 54 to separate the eye movement and brain activity components of the signal. Eye movements are identified within the eye movement channel at step 55. The brain activity data is processed at step 56 using a frequency domain representation to identify the dominant frequencies. There are other ways of finding the dominant frequencies. The coefficients identifying the dominant frequencies are fed at step 57 into a neural network which, in the same way as in EP-A-0773504, generates probabilities that the subject is in one of a number of sleep stages.

If the probabilities generated by the neural network indicate a high probability of sleep stage 1 or stage REM at step 58 then the eye movement data is used to distinguish between them at step 67. The distinction between stage 1 and REM sleep is possible because of the use of the EOG signal. This signal can be used to distinguish between these two types because when the subject is in REM sleep it carries low frequency eye movement structure not present during stage 1 sleep.

The probabilities of each sleep stage are combined in step 68 to form a measure of the depth of sleep. These results are then stored at step 59 and they may be displayed in any desired fashion. In the case of a vigilance monitor, the vigilance level is monitored at step 64, and if it drops below a predetermined threshold, a warning message is generated at 65. This may be used to alarm the subject, or may be stored for a later analysis.

As mentioned, above, it is also possible for the microprocessor to obtain from the signals a measurement of the heart rate because there is a breakthrough into the EOG channel of an ECG signal.

FIG. 5 to 10 show recordings from two subjects of signals from a variety of electrodes. FIGS. 5 and 6 show a full nights sleep with the time in seconds along the horizontal axis. The top plot (1) in each figure shows the expert scoring in a standard R&K hypnogram in which:—M=Movement; W=Wake; and R=REM. The second and third plots show the right-side and left-side outputs EOG in a form in which the value +1 corresponds to wake, 0 corresponds to REM/light sleep and −1 corresponds to deep sleep. Although not visible in these second and third plots of FIGS. 5 and 6, the present invention allows the REM to be distinguished from light sleep by displaying these two different sleep stage types in a distinguishing manner by, for example, displaying REM sleep in one colour and light sleep in another colour.

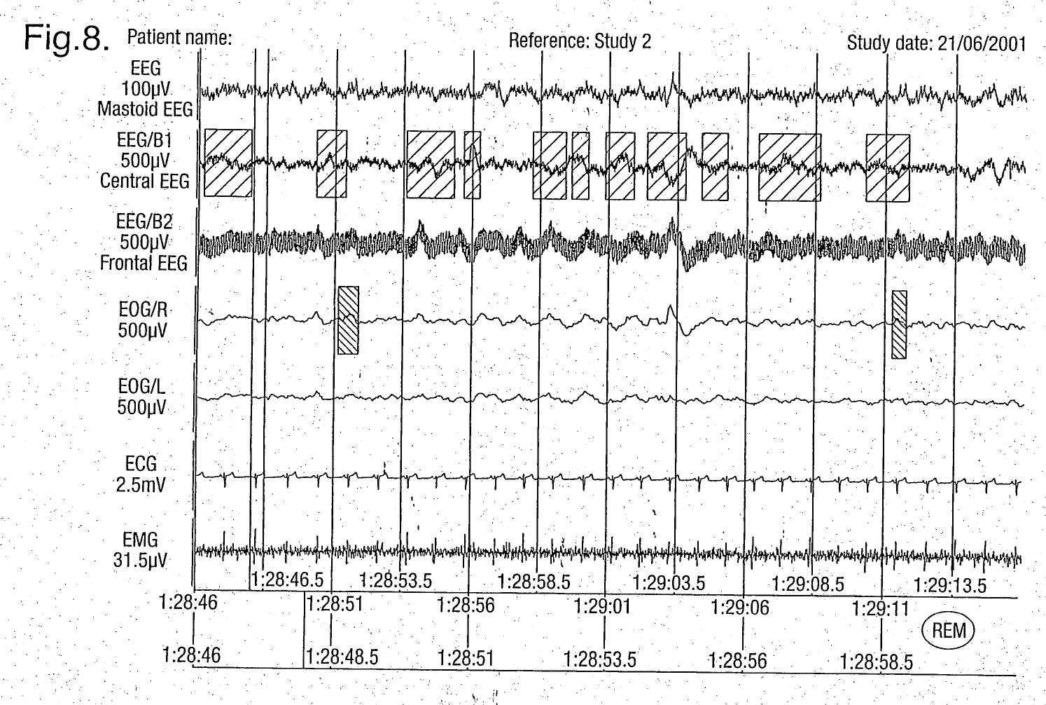

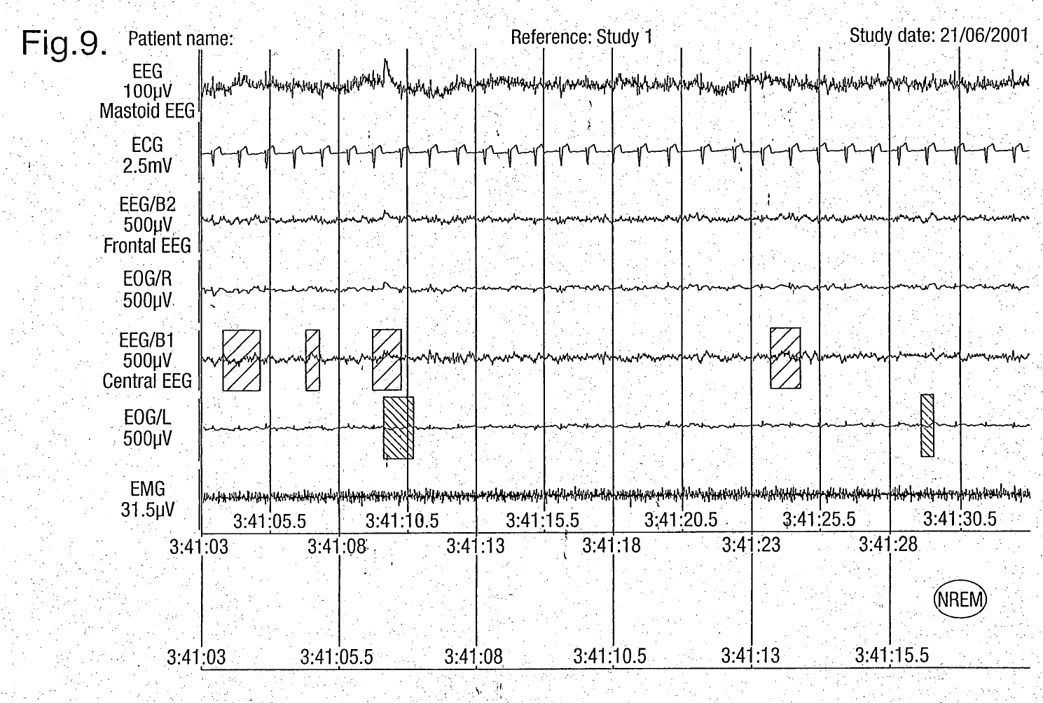

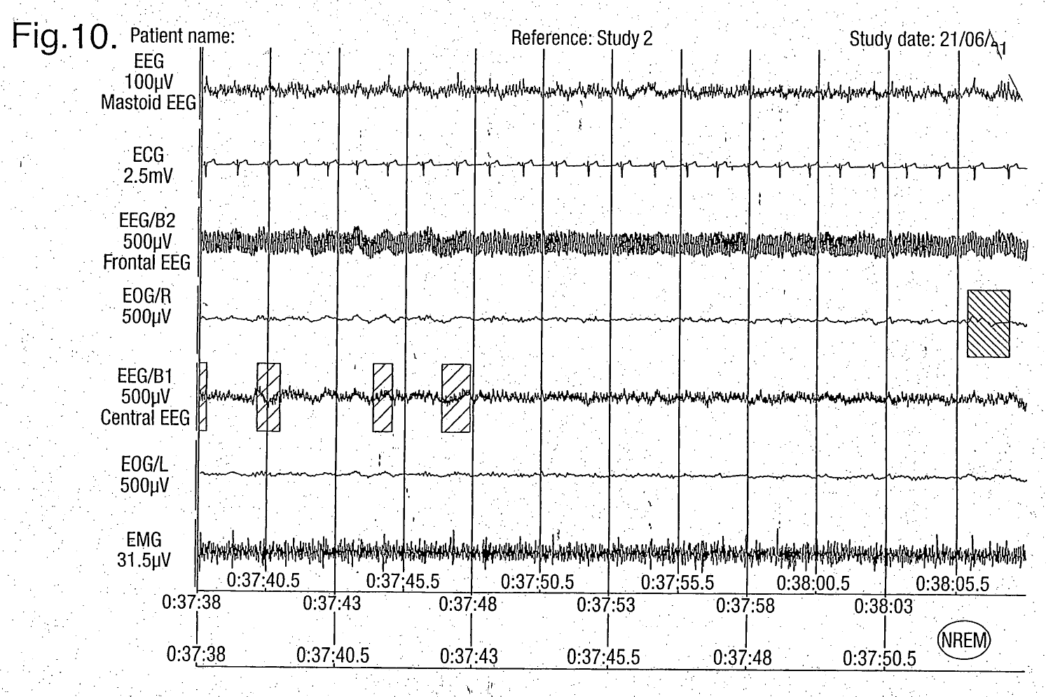

FIG. 7 to 10 show approximately 30 seconds of raw data from seven channels, including the left and right EOG channels. FIG. 7 shows a period of REM sleep for a first subject, and FIG. 8a period of REM sleep for a second subject. In both cases the low frequency component on the EOG channels can be seen. This is particularly noticeable if compared with FIGS. 9 and 10 which show 30 seconds of data for each subject respectively which correspond to a period of non-REM sleep. It will be seen that the low frequency eye movement structure is not present on the EOG left and right channels for either subject.

The fact that the device is compact and simple, and uses only a few electrodes, means that the accuracy of diagnosis of sleep disorders is improved because the device itself does not interfere so much with the patient's sleep. Further, it allows a first screening of patients with sleep disorders to be undertaken before the patients are referred to a specialist practitioner or sleep clinic.

The quality of sleep is also of considerable interest to elite sportsmen and women and again the ease of use of the device means that sleep monitoring is a realistic possibility for such people.

In a further embodiment the device may be used as an alarm clock which is sensitive to the quality of sleep which has been achieved and the sleep stage of the subject. Thus the device can wake the subject up after a certain number of hours of good quality sleep or alternatively wake them only during a period of light sleep or REM after a certain number of hours in bed.

As indicated above the invention is also applicable to vigilance monitoring. Vigilance research involves determining when an awake subject, normally with open eyes, starts to move from their wakeful state towards light sleep with a loss of vigilance. Many researchers have studied vigilance but no systems for alerting a loss of vigilance are in common use. The difficulty with, using EEG data to score vigilance levels is that some people can operate adequately when they have, significant quantities of theta activity (as shown in FIG. 2B) in the EEG and might otherwise be scored as being in Stage 1 sleep. Conversely, some operators will lose vigilance while having a majority of beta activity (as shown in FIG. 2A) on their EEG.

The best approach for screening the vigilance level of an operator is to take video recordings of the operator's face, and in particular their eyes, and to have this recording scored by an expert examiner. The examiner is trained to look for signs of fatigue on the recording. These signs are reflected by behavioural patterns such as “droopy eyes”, “slow blinks”, “head nods”, “eyes shut for seconds” and “yawns”. The main drawback of a video system is that when the operator turns their head, the signal is lost. Some investigators have tried to circumvent this issue by mounting the video camera on the operator's head although this is not a particularly practical solution.

Using the same (preferably single) EOG channel, as above for distinguishing between REM and Light Sleep, low frequency eye movement data will be produced on the EOG signals when the eyes are moving and the eyelids are open. Signals are also detected when the eyelids open and close. The neural network is trained to distinguish between a ‘fast’ blink or movement of the eyes and signs of fatigue such as a slower-closing of the eyes or rolling of the eyes, or long periods of no eye movement if the operator is staring into the distance, based on low frequency eye movement data on the EOG trace.

This low frequency eye movement data is used in conjunction with the standard brain wave activity on the EOG signal to produce an accurate vigilance monitor. The data may be processed by a neural network. Because of subject-to-subject differences in vigilance levels the training of the neural network may be subject specific where necessary.

1. Insomnia monitoring apparatus comprising:
one or more electrodes to obtain an electro-oculogram (EOG) signal from a subject over a period of epochs, the signal being related to the sleep stage type being experienced by the subject; and
a processor adapted to analyse the electro-oculogram signal and assign a sleep stage type to each epoch based on the electro-oculogram signal.

2. Insomnia monitoring apparatus according to claim 1 wherein the processor is adapted to generate a hypnogram from the assigned sleep stage types, to analyse the hypnogram to generate a summary index of sleep quality over the period of epochs; and wherein the apparatus further comprises a display for displaying the summary index of sleep quality.

3. Vigilance monitoring apparatus comprising:
one or more electrodes to obtain an electro-oculogram (EOG) signal from a subject over a period of epochs, the signal being related to the wakefulness stage type being experienced by the subject; and
a processor adapted to analyse the electro-oculogram signal and assign a wakefulness stage type to each epoch based on the electro-oculogram signal.

4. Vigilance monitoring apparatus according to claim 3 further comprising a wakefulness stage type monitor for monitoring the assigned wakefulness stage types to determine whether they meet predetermined criteria which represent a lowered level of vigilance; and
a message generator responsive to the wakefulness stage type monitor to generate a message when the predetermined criteria are met.

5. Insomnia or vigilance monitor apparatus according to claim 1 wherein the hypnogram or assigned wakefulness stage types are generated from the electro-oculogram signal only.

6. Insomnia or vigilance monitor apparatus according to claim 1 wherein the EOG signal is obtained from at least one of the left and right EOG sites, referenced to at least one of the left and right mastoid sites and a quiet site on the scalp.

7. Insomnia or vigilance monitor apparatus according to claim 1 wherein the EOG signal is obtained from one of the left and right EOG sites referenced to the corresponding neighbouring mastoid site.

8. Insomnia or vigilance monitor apparatus according to claim 1 wherein the processor distinguishes between the sleep stage types REM and light sleep.

9. Insomnia or vigilance monitor apparatus according to claim 1 wherein the processor distinguishes between the sleep stage types wake, REM, light sleep, and deep sleep stage.

10. Insomnia or vigilance monitor apparatus according to claim 1 wherein the processor comprises a trained artificial neural network.

11. Insomnia or vigilance monitor apparatus according to claim 1 wherein the monitor comprises a single portable unit.

12. Insomnia monitor apparatus according to claim 2 wherein the summary index comprises a Sleep Efficiency Index or an indication of the periodicity of the sleep/wake continuum.

13. Insomnia or vigilance monitor apparatus according to claim 1 wherein the processor is adapted to analyse the electro-oculogram signal to derive a heart rate measurement therefrom.

14. A method of sleep or vigilance monitoring, characterised by: obtaining at least one electro-oculogram (EOG) signal from a subject and performing sleep or vigilance analysis on the at least one EOG signal.

15. A method according to claim 14 comprising attaching first and second electrodes to respectively an EOG site and a reference site and monitoring the differential electrical signals between the two electrodes.

16. A method according to claim 15 wherein the reference site is the mastoid site neighbouring the EOG site.

17. A method according to claim 14 wherein only said EOG signal is used for performing said sleep or vigilance analysis.

18. A method according to claim 14 wherein REM sleep is distinguished from Light Sleep, deep sleep and an awake state using a single channel of EEG data.

19. A method according to claim 18 wherein the EEG data is recorded from an EOG site.

20. A method according to claim 14 further comprising deriving a heart rate measurement from said EOG signal.

21. A method according to claim 14, wherein:
the EOG signal is obtained from the subject over a period of epochs, the signal being related to the sleep or wakefulness stage type being experienced by the subject; and
said analysis comprises assigning a sleep or wakefulness stage type to each epoch.

22. A method of insomnia monitoring according to claim 21 further comprising:
analysing assigned sleep stage types to generate a summary index of sleep quality over the period of epochs; and
displaying the summary index of sleep quality.

23. A method of vigilance monitoring according to claim 21 further comprising:
monitoring the assigned wakefulness stage types to determine whether they meet predetermined criteria representing a lowered level of vigilance; and
generating an alarm message when the predetermined criteria are met.

24. A method according to claim 14, wherein said analysis is performed by a trained artificial neural network.

25. An alarm clock comprising insomnia monitoring apparatus according to claim 1 and further comprising an alarm controller for triggering an alarm in accordance with predefined criteria based on sleep stage types assigned by said processor.

26. An alarm clock in accordance with claim 25 wherein said predefined criteria comprise the condition that a predefined time has elapsed in one or more predetermined ones of the sleep stage types.

27. An alarm clock in accordance with claim 26 wherein said one or more predetermined ones of the sleep stage types include at least one of deep sleep stage types 1 to 4.

28. An alarm clock in accordance with claim 25 wherein said predefined criteria comprise the condition that the currently assigned sleep stage type is a predetermined one of the sleep stage types.

29. An alarm clock in accordance with claim 28 wherein said predetermined one of the sleep stage types includes at least one of light sleep and REM sleep.
